(19) United States
(12) Patent Application Publication
(10) Pub. No.: US 2009/0254117 A1
(43) Pub. Date: Oct. 8, 2009

(54) Venous Filter with Detachable Anchors

(76) Inventor: Robert L. Pakter, Tucson, AZ (US)

Correspondence Address: Lawrence F. Rozsnyai, 398 El Paseo, Millbrae, CA, 94030, US

(21) Appl. No.: 12/417,990

(22) Filed: Apr. 3, 2009

Related U.S. Application Data
(60) Provisional application No. 61/123,043, filed on Apr. 3, 2008.

(51) Int. Cl. A61M 29/00

(52) U.S. Cl. 606/200

(57) ABSTRACT

A venous filter including anchors that detach from the struts and that remain attached to the vein wall after retrieval of the other parts of the filter is described. Anchors may be detachably connected to struts of the filter via a detachably connected ridge and groove, a detachably connected clip and ridge, a friction fit, a detachable locking pin connection, or a combination thereof.

### CROSS-REFERENCE TO RELATED APPLICATION

This application claims priority under 35 U.S.C. § 119 to U.S. Provisional Patent Application 61/123,043 filed Apr. 3, 2008, the entire contents of which are incorporated herein by reference.

### TECHNICAL FIELD

This application relates to venous filters, and more particularly to venous filters having anchors detachably connected to struts, the anchors detaching from the struts and remaining attached to the vein upon retrieval of the other parts of the filter.

### BACKGROUND

Venous filters are used to capture potentially fatal pulmonary emboli or other thrombi. Venous filters are most often implanted in the vena cava, especially the inferior vena cava, since most pulmonary emboli originate from the lower body.

Currently used venous filters include the Bird's Nest and Gunther Tulip filters (Cook Incorporated, Bloomington, Ind.), the Vena Tech LGM and Vena Tech LP filters (B. Braun Medical Inc., Bethlehem, Pa.), the Simon Nitinol filter (Bard Peripheral Vascular, Tempe, Ariz.), the Titanium Greenfield and the Over-the-Wire Greenfield filters (Boston Scientific, Inc. Natick Mass.), and the TrapEase filter (Cordis Corp./Johnson & Johnson, Inc., New Brunswick, N.J.).

Fibrotic wall reactions and endothelialization occur at points where the filter contacts the venous wall after two to three weeks following implantation. For this reason, removal of venous filters after this time can result in damage to the vein wall, including bleeding, tearing or dissection of the vein, and thrombosis.

Retrievable filters have been introduced in order to reduce the amount of tissue damage to the vein during removal. Retrievable filters are very similar in appearance to permanent filters, but with modifications to the caval attachment sites and/or hooks at one end that can facilitate their removal. Retrievable filters that are currently available in the United States include the Günther Tulip and Celect (Cook Inc.), Opt Ease (Cordis Corp.), and Recovery nitinol filters (Bard Peripheral Vascular).

Even with these advances, however, damage to the vein wall and other complications may still occur upon removal.

### SUMMARY

This disclosure describes venous filters having detachable anchors.

In one aspect, the venous filter includes one or more anchoring struts and one or more anchors, wherein each anchoring strut is detachably connected to an anchor. Each anchoring strut may be detachably connected to an anchor via a detachably engaging ridge and groove, a detachably engaging clip and ridge, a friction fit, or a combination thereof. Other means for detachably connecting the anchoring struts and anchors can be easily envisaged.

In implementations, the venous filter may also include one or more filtering struts. These filtering struts may lack a detachably connected anchor. Both anchoring struts and filtering struts may filter or capture emboli or other particulates.

Implementations may include the following features: At least one of the anchors can include a hollow cylindrical portion, the hollow cylindrical portion including a ridge, and at least one of the anchoring struts can include a groove, wherein the ridge detachably engages the groove. At least one of the anchors can include a hollow cylindrical portion, the hollow cylindrical portion including a groove, and at least one of anchoring struts can include a ridge, wherein the ridge detachably engages the groove. At least one of the anchors can include a ridge, and at least one of the anchoring struts can include a clip, wherein the clip detachably engages the groove. At least one of the anchors can include a female portion, and at least one of the anchoring struts can include a male portion, wherein the female and male portions are adapted to provide a detachable friction fit. Conversely, at least one of the anchoring struts can include a hollow cylindrical portion, the hollow cylindrical portion including a ridge, and at least one of the anchors can include a groove, wherein the ridge detachably engages the groove. At least one of the anchoring struts can include a hollow cylindrical portion, the hollow cylindrical portion including a groove; and at least one of the anchors can include a ridge; wherein the ridge detachably engages the groove. At least one of the anchoring struts can include a ridge, and at least one of the anchors can include a clip, wherein the clip detachably engages the groove. At least one of the anchoring struts can include a female portion, and at least one of the anchors can include a male portion, wherein the female and male portions are adapted to provide a detachable friction fit.

Other implementations include a venous filter having one or more anchoring struts and one or more anchors, wherein each anchoring strut is detachably connected to an anchor via a plurality of detachably engaging grooves and ridges. In certain implementations, each of the anchoring struts or each of the anchors can include a hollow cylindrical portion. The hollow cylindrical portion can be tiered.

In some implementations, the struts (i.e. both anchoring and filtering struts) and anchors of the filter can have a round or near-round cross-section. Struts and anchors can be made with other cross-sections, such as rectangular, ellipsoid, oval, egg-shaped, and so forth.

In implementations, the filter, or portions thereof, can be composed of chronichrome, cobalt-chrome alloy, silicone, stainless steel, titanium, elgiloy, Nitinol, MP35N, or a combination thereof. The filter may contain an elutable therapeutic substance. Such substances include, but are not limited to, anticoagulant agents, antiplatelet agents, antifibrinolytic agents, angiogenesis factors, activated protein C, tissue plasminogen activator, prostacyclin, and vascular endothelial growth factor.

Implementations include a method of retrieving a venous filter implanted in a vein. The method includes (i) advancing a retrieval device within the vein toward the venous filter, the venous filter the venous filter comprising one or more anchoring struts and one or more anchors, each anchoring strut detachably connected to an anchor; (ii) engaging the filter with the retrieval device; (iii) applying a force to the connection regions such that each anchoring strut detaches from each anchor; and (iv) withdrawing the retrieval device to remove the anchoring struts from the vein. In implementations, the anchors may remain attached to the vein after the remainder of the filter is removed.

Other implementations include a medical device having a body and one or more anchors detachably connected to the body via a detachably engaging ridge and groove, a detachably engaging clip and ridge, a friction fit, or a combination thereof.

As with the venous filter, other means for detachably connecting anchors to the body of the device may be easily envisaged.

Implementations also include a method of retrieving a medical device implanted in a bodily tissue comprising (i) advancing a retrieval device toward the medical device, the medical device having a body and one or more anchors, each anchor detachably connected to the body at a connection region; (ii) engaging the medical device with the retrieval device; (iii) applying a force to the connection regions such that each anchor detaches from the body of the medical device; and (iv) withdrawing the retrieval device to remove the body of the medical device.

Implementations may include one of more of the following advantages. The filter (or body of the medical device) may be removed after endothelialization has occurred at or more of the anchors. The filter may be removed without causing substantial damage to the vein wall. In the case of a medical device, the body of the device may be removed without causing substantial damage to the tissue into which it was implanted.

The details of one or more embodiments are set forth in the accompanying drawings and the description below. Other features, objects, and advantages will be apparent from the description and drawings, and from the claims.

### BRIEF DESCRIPTION OF THE DRAWINGS

FIG. 1A is an interior view of a vein with an implementation of a venous filter having detachable anchors and deployed therein.

FIG. 1B is an interior view of a vein with an alternative implementation of a venous filter having detachable anchors and deployed therein.

FIG. 2A is a cross-sectional partial view of an anchor detachably connected to a strut.

FIG. 2B is a cross-sectional partial view of the anchor and strut of FIG. 2A after detachment.

FIGS. 3A-E are cross sectional partial views of alternative implementations of an anchor that may be detachably connected to a strut.

FIGS. 4A-C are interior views of a vein illustrating the retrieval of the venous filter shown in FIG. 1A.

Although the figures represent implementations of the disclosure, they are not necessarily drawn to scale, and may emphasize or exaggerate certain features to facilitate illustration and explanation.

### DETAILED DESCRIPTION

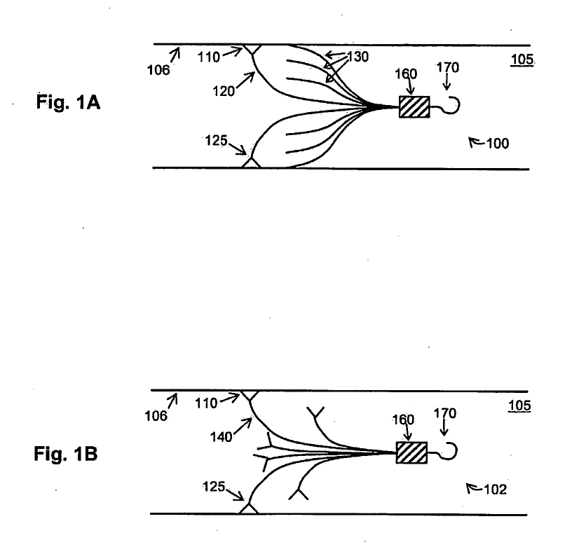

Referring to FIG. 1A, venous filter 100 is deployed in vein 105. Venous filter 100 includes at least two anchoring struts 120 leading from hub 160 at their proximal ends. One or more filtering struts 130 also lead from hub 160 at their proximal ends, contacting vein wall 106 at their distal ends. Retrieval hook 170 leads from hub 160. The distal ends of anchoring struts 120 terminate at connection regions 125, where the anchoring struts 120 are detachably connected to anchors 110.

FIG. 1B illustrates an alternative implementation of a venous filter in which all of the struts are anchoring struts. In this implementation, venous filter 102, deployed in vein 105, includes anchoring struts 140 leading from hub 160 at their proximal ends. The distal ends of anchoring struts 140 terminate at connection regions 125, where anchoring struts 140 are detachably connected to anchors 110.

FIGS. 1A-B illustrate but two exemplary implementations of the instant disclosure. Other types of venous filters, including, but not limited to the examples described above, may be equipped with anchors 110 and connection regions 125 in a manner consistent with this disclosure. In addition to venous filters, other medical devices, the removal of which may otherwise damage surrounding or contacting tissue, may also be equipped with anchors 110 and connection regions 125 in a manner consistent with this disclosure. Examples of such devices include, without limitation, stents of the trachea and smaller airways, gastrointestinal stents, and stents of the urinary tract. For example, it may be that such devices are required in situ for only a limited period of time, as may occur in the setting of malignancy, which may narrow the airway or bowel conduit sufficiently as to require implantation of a stent. With therapy however, stenoses may have resolved to sufficient degree such that the stent is no longer required, and may in fact hinder return to premorbid tissue dimensions.

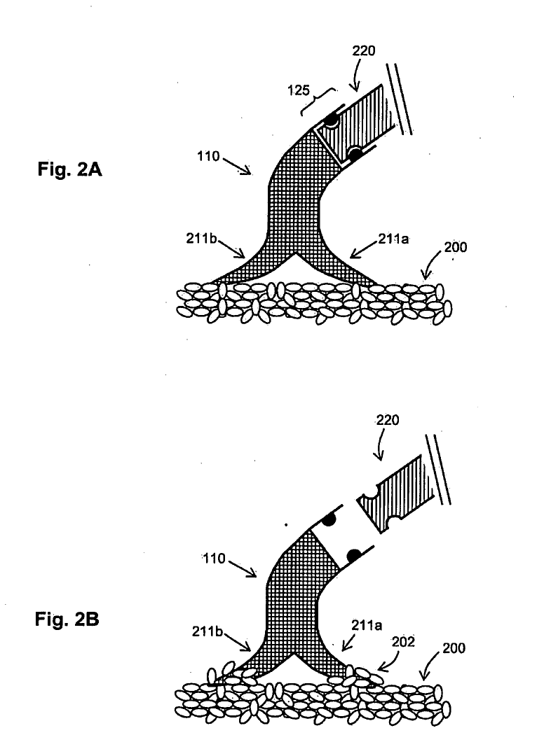

Referring to FIG. 2A, anchoring strut 220 is detachably connected to anchor 110 at connection region 125. For purposes of clarity, anchoring strut 220 may be one of the struts shown in FIGS. 1A-B, or a strut or similar structure of another type of venous filter, non-venous filter, stent, or other implantable device.

As shown in FIG. 2B, anchoring strut 220 may be detached or disconnected from anchor 110. Such detachment will occur with the application of a sufficient amount of force (e.g. medial or inward force) during removal of the filter (see also below). The force needed to detach an anchoring strut from an anchor (the “detachment force”) will typically be greater than the force applied to the filter while deployed in the vein, even under maximum theoretical clot load (prior to complete occlusion), and less than the force necessary to remove the anchor from the vein wall. Thus, when the detachment force is applied to connection regions 125 (see FIG. 2A), anchoring struts 120 (FIG. 1A) or anchoring struts 140 (FIG. 1B) will detach from anchors 110. Thus, anchors 110 may be left attached to the vein wall after the remainder of the filter (or other medical device) is removed. Standard engineering and fabrication methods known to those with skill in the art can be used to select the dimensions and tolerances of the connection regions to precisely control the amount of force within these limits. Moreover, the detachment force will be selected so as to not occur physiologically. While certain forces (medial and others) may occur unnaturally or pathologically, for example in the setting of blunt trauma, surgery, or with malignant compression of the inferior vena cava, the detachment force can be selected to exceed the forces applied in a vast majority of such events.

Referring again to FIG. 2A, anchor 110 includes hooks 211a and 211b that contact vein wall 200. One hook, shown as 211a, may be cephalad (pointing towards the head) and another, shown as 211b, may be caudal (pointing towards the feet). The two hooks may be the same size, as shown, or may be of different sizes. It will be appreciated that hooks 211a-b may independently assume various shapes (e.g. curved, straight, pointed, flat, etc.) such as those shapes employed in various venous filters or other implantable medical devices. Hooks 211a-b may also include one or more barbs, not shown, to further increase adhesion to and/or penetration into vein wall 200. Also, although anchor 110 is shown with two hooks, 211a-b, anchors consistent with this disclosure may include one hook or more than two hooks.

Anchor 110 (as well as other parts of the filter) may be designed to reduce turbulence and to promote normal blood flow. For example, anchors 110, anchoring struts 120 (or filter struts 140), and other parts of the filter may have a round or near-round cross-section. The anchors and struts (as well as other parts of the filter) may be made of and/or coated with various materials consistent with implantable devices to further control the detachment force. Such materials include, but are not limited to, chronichrome, cobalt-chrome alloy, polytetrafluoroethylene (e.g. Teflon®), silicone, stainless steel, titanium, elgiloy, Nitinol, and MP35N. The anchors and struts (as well as other parts of the filter) may also contain one or more elutable therapeutic substances (e.g. as a coating, an interlayer, etc.). Examples of elutable therapeutic substances include, without limitation, anticoagulant agents, antiplatelet agents, antifibrinolytic agents, angiogenesis factors, activated protein C, tissue plasminogen activator, prostacyclin, and vascular endothelial growth factor.

FIG. 2B-A also illustrate endothelialization of the anchor. At the time of deployment, shown in FIG. 2A, hooks 211a and 221b of anchor 110 are in contact with vein wall 200. As shown in FIG. 2B, proliferating intimal cells 202 may begin to accumulate at the point of contact (“endothelialization”), typically at about two to three weeks following deployment of the filter. As described above, such endothelialization increases the likelihood and degree of damage to the vein wall if anchors and hooks are removed from the vein wall when the filter is retrieved. In certain implementations, connection region 125 will be located sufficiently far from the vein wall so that any endothelialization that may occur on or at the connection region will not substantially interfere with disconnection of the anchor from the strut. Any such substantial interference could result, for example, in increasing the detachment force to above that force needed to remove the anchors from the vein walls. Connection region 125 will typically also be located sufficiently close to vein wall 200 so that after retrieval, anchors remaining attached to the vein wall will not substantially interfere with normal blood flow.

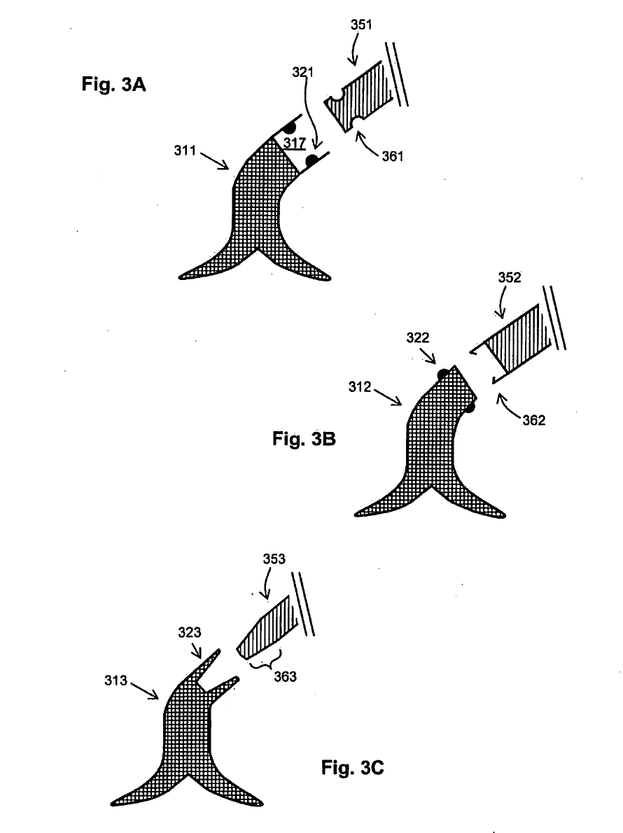

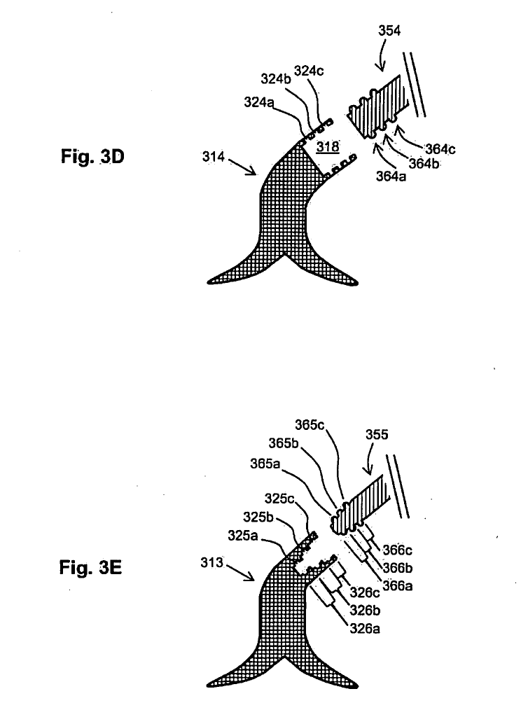

As shown in FIGS. 3A-E, an anchoring strut may be detachably connected to an anchor in a variety of ways. In certain implementations, as illustrated in FIG. 3A, anchor 311 is provided with a hollow cylindrical region 317 including internal ridge 321 located along an internal circumference of cylindrical region 320. Anchoring strut 351 is provided with a peripheral grove 361. Peripheral grove 361 may detachably engage internal ridge 321, detachably connecting anchoring strut 351 to anchor 311. In this example, the ridge and groove are shown with a rounded cross-section, however they may have cross sections that are squared-off, or of other shapes. Also, the arrangement of the detachably engaging ridge and groove may be inverted so that the anchoring strut includes the ridge and the anchor includes the groove. Multiple ridges and grooves may be used. Other similar arrangements may be easily envisaged.

FIG. 3B illustrates an alternative detachable connection. In this implementation, anchor 312 includes external ridge 322. Anchoring strut 352 includes clip 362, which can detachably engage ridge 322. In this example, the ridge has a squared-off (e.g. rectangular) cross-section, but may have a cross-section of a different shape as described above. The arrangement of the detachably engaging clip and ridge can also be inverted with respect to placement on the anchoring strut and anchor, as described above.

FIG. 3C illustrates a detachable friction-fit connection. In this implementation, anchor 313 includes female end 323. Anchoring strut 353 includes tapered male end 363. Male end 363 and female end 323 are adapted to provide a detachable friction fit connection between the anchoring strut and the anchor.

FIG. 3D illustrates a detachable “locking pin” connection. In this implementation, anchor 314 is provided with a hollow cylindrical region 318 including peripheral grooves 324a, 324b, and 324c. Anchoring strut 354 includes peripheral ridges 364a, 364b, and 364c. When a filter including this connection is deployed in a vessel, ridges 364a, 364b, and 364c will detachably engage grooves 324a, 324b, and 324c respectively, detachably connecting anchoring strut 354 to anchor 314. The multiple ridges and grooves may provide additional protection against unwanted detachment of the anchoring struts from the anchors, for example, in patients expected to present heavy clot loads on the filter. For example, in addition to detachably engaging groove 324a, ridge 364a may detachably engage groove 324b or 324c. Similarly, in addition to detachably engaging groove 324b, ridge 364b may detachably engage groove 324c. The ridges and grooves may be made the same size and shape, or can be made of different sizes and shapes to further control the detachment force and degree of protection against unwanted detachment. While the implementation illustrated in FIG. 3D shows three interlocking ridges and grooves, other implementations may include any number of interlocking grooves and ridges, for example as little as two or as many as five interlocking ridges and grooves may be used.

FIG. 3E illustrates the detachable locking pin connection shown in FIG. 3D, but with tiered regions. In this implementation, anchor 315 is provided with a hollow cylindrical region 319 containing tiers 326a, 326b, and 326c. The tiers result in cylindrical region 319 having different diameters (cross-sections not shown). For example, the diameter of tier 326a is smaller than the diameter of tier 326b, which is in turn smaller than the diameter of tier 326c. Located on tiers 326a, 326b, and 326c, respectively, are grooves 325a, 325b, and 325c. Anchoring strut 355 includes tiers 366a, 366b, and 366c. Located thereon, respectively, are ridges 365a, 365b, and 365c. When a filter including this connection is deployed in a vessel, ridges 365a, 365b, and 365c will detachably engage grooves 325a, 325b, and 325c respectively, detachably connecting anchoring strut 355 to anchor 315. Tiers 366a, 366b, and 366c will also mate with tiers 326a, 326b, and 326c. The height/depth of the tiers may be smaller relative to the height and depth of the ridges and grooves, than as depicted in FIG. 3E.

The detachable connections illustrated in FIGS. 3A-E are exemplary, and alternative ways or means of detachably connecting an anchor to an anchoring strut may also be used. Multiple connection ways or means may also be used. For example, a ridge-and-groove connection may be combined with a friction-fit connection, or a locking pin connection can be combined with a fiction-fit connection. Other detachable connections and combinations consistent with this disclosure can be easily envisaged.

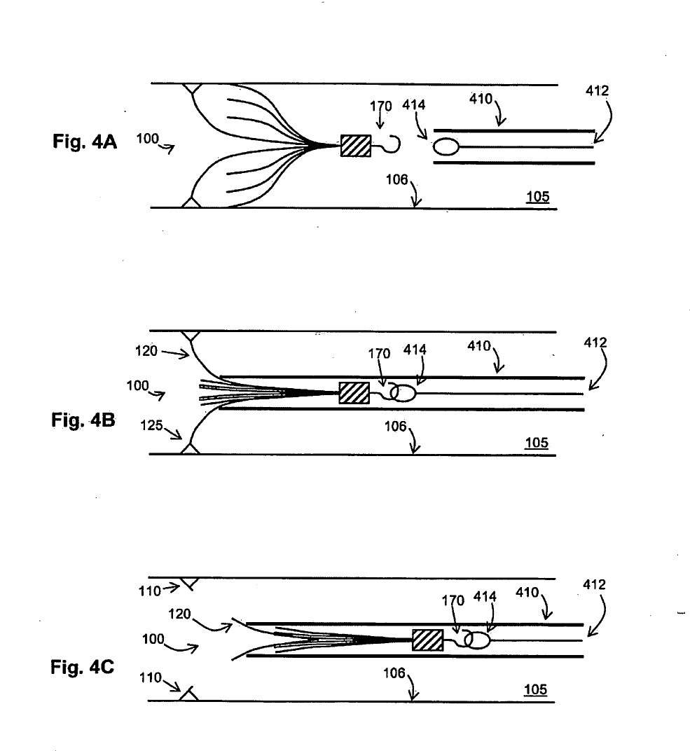

FIGS. 4A-C illustrate an exemplary method of retrieval of the implementation of venous filter 100 shown in FIG. 1A. This procedure will typically be performed by a medical specialist such as a suitably trained physician. A retrieval device will be introduced percutaneously into the vein. For example, to retrieve a filter implanted in the inferior vena cava, a retrieval device may be introduced in the jugular vein, although other points of entry may also be used. As shown in FIG. 4A, the retrieval device introduced into vein 105 includes sheath 410, snare 412, and loop 414. The specialist will manipulate loop 414 to engage retrieval hook 170, as shown in FIG. 4B. Sheath 410 will then be advanced over filter 100, holding in place or temporarily extending snare 412 and loop 414 so as not to prematurely exert a medial force on connection regions 125. Once sheath 410 is sufficiently positioned so as to engage or partially encompass anchoring struts 120, a force (e.g. a medial force) can be applied along anchoring struts 120 to connection regions 125, for example by a quick pull of snare 412 and loop 414. As shown in FIG. 4C, when this force equals the detachment force, anchoring struts 120 will detach from anchors 110, allowing the remainder of filter 100, including anchoring struts 120, to be removed from the vein. Anchors 110 may remain attached to vein wall 106. Other methods of retrieval in which a force is used to disconnect the struts from the anchors may also be envisaged.

A number of embodiments of the invention have been described. Nevertheless, it will be understood that various modifications may be made, including modifications to shape, size, and arrangement of parts, without departing from the spirit and scope of the invention, including the aforementioned nonvascular applications and others. Accordingly, other embodiments are within the scope of the following claims.

What is claimed is:

1. A venous filter comprising
one or more anchoring struts;
one or more anchors; and
a means for detachably connecting each anchoring strut to an anchor.

2. A venous filter comprising
one or more anchoring struts; and
one or more anchors;
wherein each anchoring strut is detachably connected to an anchor via a detachably engaging ridge and groove, a detachably engaging clip and ridge, a friction fit, or a combination thereof.

3. The filter of claim 2 wherein at least one of the anchors includes a female portion, and wherein at least one of the anchoring struts includes a male portion.

4. The filter of claim 2 wherein at least one of the anchors includes a male portion, and wherein at least one of the anchoring struts includes a female portion.

5. The filter of claim 2 wherein at least one of the anchoring struts or at least one of the anchors includes a hollow cylindrical portion.

6. The filter of claim 5 wherein the hollow cylindrical portion includes a ridge or a groove.

7. The filter of claim 5 wherein the hollow cylindrical portion is tiered.

8. The filter of claim 2 wherein the anchoring struts and anchors have round or near-round cross-sections.

9. The filter of claim 2 composed of chronichrome, cobalt-chrome alloy, silicone, stainless steel, titanium, elgiloy, Nitinol, MP35N, or a combination thereof.

10. The filter of claim 2 containing an elutable therapeutic substance.

11. The filter of claim 10 wherein the elutable therapeutic substance is selected from the group consisting of anticoagulant agents, antiplatelet agents, antifibrinolytic agents, angiogenesis factors, activated protein C, tissue plasminogen activator, prostacyclin, and vascular endothelial growth factor.

12. The filter of claim 2 wherein each of the anchoring struts is detachably connected to an anchor at a connection region.

13. The filter of claim 12 wherein the connection regions are located so that while the filter is deployed in a vein, endothelialization does not substantially interfere with disconnection of the anchors from the anchoring struts.

14. The filter of claim 12 wherein the connection regions are located so that after retrieval, the anchors will not substantially interfere with normal blood flow.

15. The filter of claim 2 further comprising one or more filtering struts.

16. A method of retrieving a venous filter implanted in a vein comprising
advancing a retrieval device within the vein toward the venous filter, the venous filter comprising one or more anchoring struts and one or more anchors, each anchoring strut detachably connected to an anchor;
engaging the filter with the retrieval device;
applying a force to each anchoring strut such that each anchoring strut detaches from each anchor; and
withdrawing the retrieval device to remove the anchoring struts from the vein.

17. A medical device comprising
a body; and
one or more anchors detachably connected to the body via a detachably engaging ridge and groove, a detachably engaging clip and ridge, a friction fit, or a combination thereof.

18. A method of retrieving a body of medical device implanted in a bodily tissue comprising
advancing a retrieval device toward the medical device, the medical device having a body and one or more anchors, each anchor detachably connected to the body at a connection region;
engaging the medical device with the retrieval device;
applying a force to the connection regions such that each anchor detaches from the body of the medical device; and
withdrawing the retrieval device to remove the body of the medical device.
